(19) United States
(12) Patent Application Publication
(10) Pub. No.: US 2023/0166996 A1
(43) Pub. Date: Jun. 1, 2023

(54) High-Value Treatment System or Method for Urban Wet Garbage

(71) Applicants: Tongji University, Shanghai (CN); University of Science and Technology Beijing, Beijing (CN)

(72) Inventors: Yinguang Chen, Beijing (CN); Xiong Zheng, Beijing (CN); Xuemeng Zhang, Beijing (CN); Chuang Chen, Beijing (CN); Qunhui Wang, Beijing (CN); Ming Gao, Beijing (CN)

(21) Appl. No.: 17/445,055

(22) PCT Filed: Apr. 21, 2021
(86) PCT No.: PCT/CN2021/088587

§ 371 Date: Aug. 13, 2021

(30) Foreign Application Priority Data
Feb. 25, 2021 (CN) 202110214854.X

(51) Int. Cl. C02F 11/04; C02F 11/143; C02F 11/147; C12P 7/54

(52) U.S. Cl. CPC C02F 11/04; C02F 11/143; C02F 11/147; C12P 7/54

(57) ABSTRACT

The present invention belongs to the field of treatment of urban organic wastes, and specifically relates to a high-value treatment system or method for urban wet garbage. According to the present invention, through the steps such as oil extraction, high-efficiency hydrolysis, high-value biological conversion, simultaneous recovery of released nitrogen and phosphorus and deep utilization of residues, urban wet garbage is converted into acetic acid by high-value treatment, produced by-products including carbon dioxide and hydrogen are biologically converted into acetic acid, released nitrogen and phosphorus are recycled into slow-release fertilizers, and solid residues are used to prepare materials capable of promoting conversion of the wet garbage into acetic acid through high-value treatment. According to the present invention, not only can high-value treatment of the urban wet garbage be realized, but also produced waste gases and waste residues are recycled.

### TECHNICAL FIELD

The present invention belongs to the field of treatment of urban organic wastes, and specifically relates to a high-value treatment system or method for urban wet garbage.

### BACKGROUND

With rapid development of economy and improvement of people's material living standards, the urbanization process has been accelerated. In the past five years, the output of average urban organic wastes in China is increased by more than 10%. At the end of 2020, the annual output of urban wet garbage in China is more than 500 million tons. At present, end treatment is mainly used to treat urban wet garbage in China and mainly includes sanitary landfill and incineration. According to sanitary landfill, urban organic solid wastes are placed into a depression pool, anti-seepage materials are used to prevent the situation that pollution is caused since leachates enter the groundwater, landfill gases are exported for use or combustion, and flood intercepting ditches are dug around the site to prevent floods from entering the site. Incineration is a process that urban wet garbage with a certain heat value is subjected to appropriate thermal decomposition, combustion, melting and other reactions so that the volume of wastes is reduced and the wastes are turned into residues or molten solids. Although the phenomenon that a city is full of urban wet garbage is temporarily alleviated by using these methods, a large amount of secondary pollution is caused. For example, a large amount of landfill leachates, foul odor, dioxin, mercury emissions and other problems are caused.

According to classification, urban wet garbage mainly includes kitchen wastes, urban sludge and the like and contains a large amount of organic substances such as polysaccharides and proteins. Under the action of anaerobic microorganisms, these organic substances can be converted into a variety of products, including gaseous products such as methane and hydrogen, and liquid products such as short-chain fatty acids and lactic acid. Compared with gaseous products, liquid products such as acetic acid have a wider application range and a higher utilization value. Therefore, preparation of liquid chemicals such as acetic acid from urban wet garbage is an important research content of high-value treatment in recent years. According to the basic principle, polysaccharides and proteins in wet garbage are hydrolyzed by enzymes to produce hydrolysates such as monosaccharides, amino acids and long-chain fatty acids; and then under the action of acid-producing microorganisms, these hydrolysates are biologically converted into acetic acid and other substances. However, carbon dioxide, hydrogen and other gases are also produced during biological conversion. In addition, serious secondary pollution is caused if solid residues obtained after biological conversion are discharged into the environment without treatment.

### SUMMARY

In view of the shortcomings of the prior art, an objective of the present invention is to provide a high-value treatment system or method for urban wet garbage so as to solve the problems in the prior art.

In order to achieve the objective above and other related objectives, the present invention is achieved through the following technical solutions.

An objective of the present invention is to provide a high-value treatment system or method for urban wet garbage, and the method includes the following steps:

1) mixing the urban wet garbage with water and performing oil extraction to obtain an oil-extracted mixture;

2) mixing the oil-extracted mixture with an alkali for performing a hydrolysis reaction to obtain a hydrolysate;

3) performing anaerobic culture on the hydrolysate and a first acetic acid-producing sludge, collecting a produced gas and performing solid-liquid separation after culture is completed to obtain a first liquid and a first solid;

4) introducing the produced gas into a mixture of municipal sewage and a second acetic acid-producing sludge for performing anaerobic culture and solid-liquid separation to obtain a second liquid and a second solid; and

5) mixing the first liquid with the second liquid, adding a magnesium salt to adjust a pH value, and performing stirring and solid-liquid separation, where a precipitate is a fertilizer containing nitrogen and phosphorus, and a supernatant liquid is a liquid containing acetic acid; mixing the first solid, the second solid and humic acid, and performing drying to obtain a third solid,

where, the first acetic acid-producing sludge is an acclimated sludge capable of converting glucose into acetic acid; and

the second acetic acid-producing sludge is an acclimated sludge capable of converting carbon dioxide and hydrogen into acetic acid.

According to the present invention, a mixture of urban wet garbage and water is heated to 65° C. and then added into a three-phase oil extractor for oil extraction, an oil phase is separated, and an oil-extracted mixture is obtained, where the oil content of the oil-extracted mixture is lower than 3%.

The urban wet garbage in the present invention refers to food wastes, leftovers, expired food, melon and fruit rinds and seeds, flowers and green plants, Chinese medicine dregs and other perishable biomass domestic wastes.

Since microbial acclimation needs to be performed in a liquid phase system, the municipal sewage is used to replace tap water in the present invention so that consumption of water resources can be reduced. Main properties of the municipal sewage are as follows: a pH value is 6.7-7.3, soluble COD is 80-140 mg/L, soluble ammonia nitrogen is 17-31 mg/L, and soluble orthophosphate is 3.3-5.5 mg/L.

The sludge in the present invention refers to surplus sludge of a sewage treatment plant, a pH value of the sludge is 6.0-7.0, a concentration of a suspension is 900-10400 mg/L, and a molar ratio of carbon to nitrogen is 5.0-7.5.

Preferably, an acclimation process of the first acetic acid-producing sludge includes the following steps: adding glucose into a mixture of sludge and municipal sewage, and performing anaerobic fermentation at a pH of 6-11 and a temperature of 20° C. to 80° C. to obtain the first acetic acid-producing sludge.

More preferably, the acclimation process of the first acetic acid-producing sludge includes three periods;

further preferably, in the first period, a content of solids in the mixture is 3800-4500 mg/L.

Further preferably, in the first period, based on a total volume of the sludge, the municipal sewage and the glucose, a concentration of glucose is 600 mgCOD/L to 1000 mg/L. More specifically, the concentration of the glucose is 800 mgCOD/L.

Further preferably, the first period is 3-7 days. More specifically, the culture time is 5 days.

Further preferably, in the second period, the concentration of the glucose is maintained to be 1000-1400 mgCOD/L per day; and more specifically, the concentration of the glucose is maintained to be 1200 mgCOD/L per day.

Further preferably, the second period is 8-12 days. More specifically, the culture time is 10 days.

Further preferably, in the third period, the concentration of the glucose is daily increased by 80-100 mgCOD/L. More specifically, the concentration of the glucose is daily increased by 100 mgCOD/L.

Further preferably, in the third period, acetic acid is also added, and a concentration of the acetic acid is maintained to be 30-70 mgCOD/L per day. More specifically, the concentration of the acetic acid is maintained to be 50 mgCOD/L per day.

Further preferably, the third period is 30-35 days. More specifically, the culture time is 34 days.

The entire anaerobic culture cycle is 50-52 days, the pH value is 6-11, and the culture temperature is 20° C. to 80° C.

Preferably, an acclimation process of the second acetic acid-producing sludge includes the following steps: introducing hydrogen and carbon dioxide into a mixture of sludge and municipal sewage, and performing anaerobic fermentation at a pH of 5-9 and a temperature of 20° C. to 50° C. to obtain the second acetic acid-producing sludge.

More preferably, a concentration of solids in the mixture is 3500-5500 mg/L.

More preferably, a molar ratio of hydrogen to carbon dioxide is (0.5-3.5):1.

Further preferably, a molar ratio of hydrogen to carbon dioxide is 2:1.

Preferably, in step 1), a particle size of the urban wet garbage is 0.1-1 mm.

Preferably, in step 1), a content of solids in a mixture formed by mixing the urban wet garbage with water is 20-180 g/L.

More preferably, the content of solids in the mixture formed by mixing the urban wet garbage with water is 50-160 g/L.

Preferably, in step 2), the alkali is sodium hydroxide, and conditions of the hydrolysis reaction are that a pH value is 8-12 and a temperature is 5° C. to 80° C.

More preferably, the conditions of the hydrolysis reaction are that the pH value is 9-11 and the temperature is 45° C. to 80° C.

Preferably, in step 2), the hydrolysis reaction time is 1-96 hours.

More preferably, the hydrolysis reaction time is 24-72 hours.

Preferably, in step 3), a volume ratio of the first acetic acid-producing sludge to the urban wet garbage is (6-10):100.

More preferably, the volume ratio of the first acetic acid-producing sludge to the urban wet garbage is (7-9):100.

Preferably, in step 3), conditions of anaerobic culture are that a pH value is 6-12 and a temperature is 20° C. to 80° C.

More preferably, conditions of anaerobic culture are that the pH value is 8-11 and the temperature is 30° C. to 60° C.

Preferably, in step 3), the anaerobic culture time is 1-12 days.

More preferably, the anaerobic culture time is 6-12 days.

Preferably, in step 3), the third solid is also added into the hydrolysate and the first acetic acid-producing sludge.

More preferably, an added amount of the third solid is not more than 70% of a dry weight of the first acetic acid-producing sludge.

Further preferably, the added amount of the third solid is 30% to 60% of the dry weight of the first acetic acid-producing sludge.

Preferably, in step 4), based on a total volume of the mixture, a concentration of the second acetic acid-producing sludge is 500-7000 mg/L.

Preferably, in step 4), conditions of anaerobic culture are that a pH value is 6-8.

More preferably, conditions of anaerobic culture are that the pH value is 7.

Preferably, in step 5), the magnesium salt is magnesium chloride.

Preferably, in step 5), after the magnesium salt is added, an ammonia nitrogen salt and/or a phosphate salt is also added;

more preferably, the ammonia nitrogen salt is ammonium chloride, and the phosphate salt is sodium phosphate.

More preferably, in step 5), based on a volume of a mixture formed after ammonia nitrogen and/or a phosphate salt are/is added, a molar ratio of magnesium ions to ammonium ions to phosphate ions is 1:1:1.

Preferably, in step 5), a pH value is 8-10, and the stirring time is 5-50 minutes.

More preferably, the pH value is 9-10, and the stirring time is 20-50 minutes.

Preferably, in step 5), a molar ratio of hydrogen to carbon in humic acid is (0.8-1.0):1, and an added amount of humic acid is 10% to 100% of a total dry weight of the first solid and the second solid.

More preferably, the added amount of humic acid is 20% to 60% of the total dry weight of the first solid and the second solid.

Preferably, in step 5), a drying temperature is 20° C. to 120° C.

More preferably, the drying temperature is 40° C. to 80° C.

According to the present invention, through the steps such as high-efficiency hydrolysis pretreatment, directional biological conversion, simultaneous recovery of released nitrogen and phosphorus and deep utilization of residues, urban wet garbage is converted into acetic acid by high-value treatment, produced by-products including carbon dioxide and hydrogen are biologically converted into acetic acid, released nitrogen and phosphorus are recycled into slow-release fertilizers, and high-value conversion of the urban wet garbage can be promoted by using solid residues.

Compared with existing technologies, the present invention has the following beneficial effects:

By using the method of the present invention, not only can high-value treatment be performed on organic wastes contained in the urban wet garbage, but also deep recycling of waste gases and waste residues produced during high-value treatment can be performed, so that secondary pollution generated in the whole process is minimized, production of high-value products and economic benefits of the wet garbage are maximized, and requirements of sustainable development of cities and towns are met. In particular, problems such as consumption of organic resources and a large amount of secondary pollution caused when petrochemical raw materials are used to synthesize acetic acid are solved in the present invention.

### BRIEF DESCRIPTION OF THE DRAWINGS

FIG. 1 is a flowchart showing a high-value treatment method for urban wet garbage in the present invention.

### DETAILED DESCRIPTION

The following describes implementations of the present invention by using specific examples. A person skilled in the art may easily understand other advantages and effects of the present invention from the content disclosed in this specification.

Before specific examples of the present invention are further described, it should be understood that the protection scope of the present invention is not limited to the following specific examples, and terms used in examples of the present invention are used to describe the specific examples rather than limit the protection scope of the present invention. If test methods of specific conditions are not indicated in examples below, it shall be carried out in accordance with the conventional conditions or the conditions recommended by the manufacturer.

When numerical ranges are given in the examples, it should be understood that, unless otherwise specified in the present invention, two endpoints of each numerical range and any value between the two endpoints can be used. Unless otherwise defined, meanings of all technical and scientific terms used in the present invention are the same as those generally understood by a person skilled in the technical field to which the present invention belongs. In addition to specific methods, equipment and materials used in the examples, those skilled in the art can also use any methods, equipment and materials in the prior art similar or equivalent to the methods, equipment and materials described in the examples of the present invention based on the knowledge of the prior art and the description of the present invention.

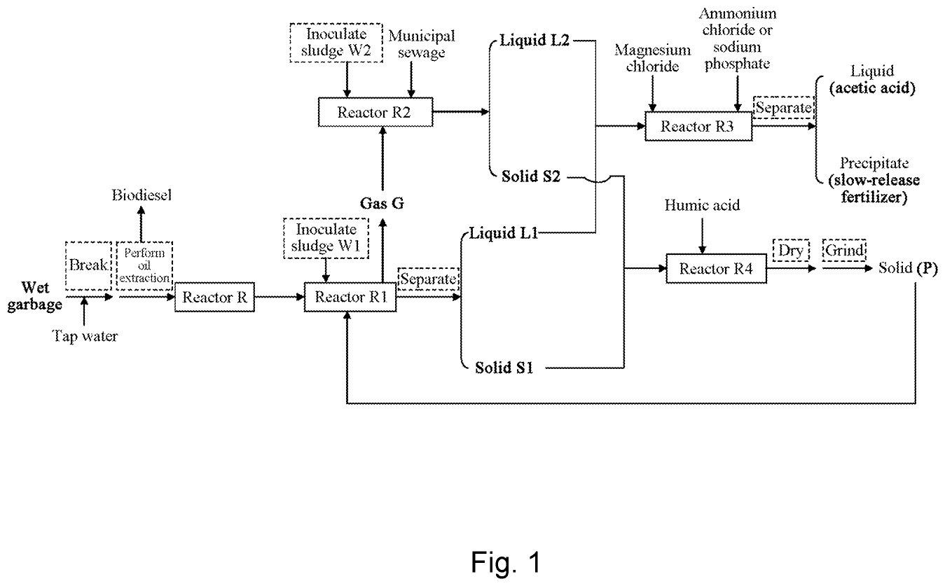

FIG. 1 is a flowchart showing a high-value treatment system or method for urban wet garbage in the present invention, and the method includes the following steps:

1) mixing the urban wet garbage with water and performing oil extraction to obtain an oil-extracted mixture, where, a particle size of the urban wet garbage is 0.1-1 mm, and a concentration of solids in the mixture formed by mixing the urban wet garbage with water is 20-180 g/L;

2) mixing the oil-extracted mixture in step 1) with an alkali for performing a hydrolysis reaction in a reactor R to obtain a hydrolysate, where, the alkali is sodium hydroxide, and conditions of the hydrolysis reaction are that a pH value is 8-12, a temperature is 5° C. to 80° C. and time is 1-96 hours;

3) performing anaerobic culture on the hydrolysate in step 2), a first acetic acid-producing sludge W1 and a third solid in a reactor R1, collecting a produced gas G and performing solid-liquid separation after culture is completed to obtain a first liquid L1 and a first solid S1, where, a volume ratio of the first acetic acid-producing sludge W1 to the urban wet garbage is (6-10):100; conditions of anaerobic culture are that a pH value is 6-12, a temperature is 20° C. to 80° C. and time is 1-12 days; and an added amount of the third solid is not more than 70% of a dry weight of the first acetic acid-producing sludge;

4) introducing the gas G produced in step 3) into a mixture of municipal sewage and a second acetic acid-producing sludge W2 for performing anaerobic culture in a reactor R2 and solid-liquid separation to obtain a second liquid L2 and a second solid S2, where, based on a total volume of the mixture, a concentration of the second acetic acid-producing sludge is 500-7000 mg/L; and conditions of anaerobic culture are that a pH value is 6-8; and

5) mixing the first liquid L1 with the second liquid L2 in a reactor R3, adding magnesium chloride, adding ammonium chloride or sodium phosphate as needed to make a molar ratio of magnesium ions to ammonium ions to phosphate ions in a solution reach 1:1:1 to adjust a pH value, and performing stirring and solid-liquid separation, where a precipitate is a fertilizer containing nitrogen and phosphorus, and a supernatant liquid is a liquid containing acetic acid. The pH value is 8-10, and the stirring time is 5-50 minutes. At the same time, the first solid S1, the second solid S2 and humic acid are mixed and dried to obtain a third solid P. The added amount of humic acid is 10% to 100% of the total dry weight of the first solid and the second solid; the drying temperature is 20° C. to 120° C.

In the present invention, the first acetic acid-producing sludge is an acclimated sludge capable of converting glucose into acetic acid. An acclimation process of the first acetic acid-producing sludge includes the following steps: adding glucose into a mixture of sludge and municipal sewage, and performing anaerobic culture at a pH of 6-11 and a temperature of 20° C. to 80° C. to obtain the first acetic acid-producing sludge.

In the present invention, the second acetic acid-producing sludge is an acclimated sludge capable of converting carbon dioxide and hydrogen into acetic acid. An acclimation process of the second acetic acid-producing sludge includes the following steps: introducing hydrogen and carbon dioxide into a mixture of sludge and municipal sewage, and performing anaerobic fermentation at a pH of 5-9 and a temperature of 20° C. to 50° C. to obtain the second acetic acid-producing sludge.

Compared with traditional methods, the yield of acetic acid can be increased by at least 157% by using the high-value treatment method for urban wet garbage in the present invention to treat urban wet garbage.

The pH values in examples of the present application are all achieved by adjusting with 10 mol/L sodium hydroxide.

### Example 1

In this example, a method for preparing a first acetic acid-producing sludge and a second acetic acid-producing sludge includes the following steps:

An acclimation process of the first acetic acid-producing sludge includes the following steps: adding surplus sludge of a sewage treatment plant and municipal sewage into a biological acclimation reactor for mixing, where the content of solids in a mixture of the sludge and municipal sewage in the biological acclimation reactor is 4000 mg/L; in a first period, adding glucose to make a concentration of the glucose reach 800 mgCOD/L based on a total volume of the sludge, municipal sewage and glucose; maintaining the pH value and the temperature in the acclimation reactor to be 6 and 20° C. respectively and performing stirring for 5 days under anaerobic conditions; in a second period, namely from the 6th day, increasing the concentration of the glucose to 1200 mgCOD/L per day, adding fresh municipal sewage per day, discharging the same amount of a supernatant per day and continuously performing anaerobic stirring for 11 days; in a third period, namely from the 16th day, increasing the concentration of the glucose by 100 mgCOD/L per day, adding acetic acid at the same time to maintain the concentration of the acetic acid to be 50 mgCOD/L per day, and discharging a supernatant and sludge per day to maintain the volume of the mixture and the concentration of the sludge in the reactor same as those on the 15th day. Acclimation was performed for 48 days to obtain the first acetic acid-producing sludge.

An acclimation process of the second acetic acid-producing sludge includes the following steps: adding surplus sludge of a sewage treatment plant and municipal sewage into another biological acclimation reactor for mixing, where the content of solids in a mixture of the sludge and municipal sewage in the biological acclimation reactor is 4500 mg/L; then adding hydrogen and carbon dioxide (a molar ratio of hydrogen to carbon dioxide is 2:1), maintaining the pH value and the temperature in the acclimation reactor to be 5 and 20° C. respectively and performing stirring under anaerobic conditions; and from the 4th day, adding hydrogen and carbon dioxide per day (the molar ratio is 2:1), and discharging a supernatant per day to maintain the volume of the mixture in the reactor same as that on the 3rd day. Acclimation was performed for 43 days to obtain the second acetic acid-producing sludge.

TABLE 1
Acclimation conditions of a first acetic acid-producing sludge and a second acetic acid-producing sludge in Examples 1-14

| | First acetic acid-producing sludge | | | | | | | |
|---|---|---|---|---|---|---|---|---|
| | Sludge | | | | Second acetic acid-producing sludge | | | |
| | concentration/ | Acclimation | Acclimation | Acclimation | Sludge | Acclimation | Acclimation | Acclimation |
| | mg/L | pH | temperature/° C. | time/d | concentration/mg/L | pH | temperature/° C. | time/d |
| Example 1 | 4000 | 6 | 20 | 48 | 4500 | 5 | 20 | 43 |
| Example 2 | 3800 | 11 | 20 | 52 | 4700 | 9 | 20 | 40 |
| Example 3 | 4300 | 6 | 80 | 40 | 4500 | 5 | 80 | 35 |
| Example 4 | 4200 | 11 | 20 | 52 | 4600 | 9 | 80 | 34 |
| Example 5 | 4100 | 9 | 40 | 43 | 4400 | 5 | 20 | 43 |
| Example 6 | 4200 | 9 | 40 | 43 | 4100 | 7 | 25 | 38 |
| Example 7 | 4300 | 9 | 40 | 43 | 4300 | 9 | 80 | 34 |
| Example 8 | 4200 | 9 | 40 | 43 | 4900 | 7 | 25 | 38 |
| Example 9 | 4400 | 9 | 40 | 43 | 4500 | 7 | 25 | 38 |
| Example 10 | 4400 | 9 | 40 | 43 | 4900 | 7 | 25 | 38 |
| Example 11 | 4400 | 9 | 40 | 43 | 4900 | 7 | 25 | 38 |
| Example 12 | 4500 | 6 | 80 | 40 | 4300 | 7 | 25 | 38 |
| Example 13 | 4100 | 9 | 40 | 43 | 4900 | 9 | 50 | 36 |
| Example 14 | 4500 | 9 | 40 | 43 | 4400 | 6 | 25 | 39 |

### Example 15

In this example, a high-value treatment method for urban wet garbage by using the first acetic acid-producing sludge and the second acetic acid-producing sludge obtained in Example 1 includes the following steps:

1) mixing the urban wet garbage with a particle size of 0.1-1 mm with water to obtain a mixture, where a concentration of the urban wet garbage in the mixture is 20 g/L; and performing boiling and oil extraction to obtaining an oil-extracted mixture;

2) placing the oil-extracted mixture in step 1) in a hydrolysis reactor R for a hydrolysis reaction to obtain a hydrolysate, where conditions for hydrolysis are that a pH is 8, a temperature is 5° C. and time is 1 hour;

3) placing the hydrolysate obtained in step 2) and the first acetic acid-producing sludge prepared in Example 1 in a reactor R1, adding a third solid for performing anaerobic culture, collecting a produced gas G and performing solid-liquid separation after culture is completed to obtain a first liquid L1 and a first solid S1, where, an added amount of the first acetic acid-producing sludge is 8% of the volume of the urban wet garbage; conditions of anaerobic culture are that a pH value is 6, a temperature is 20° C. and time is 1 day; and an added amount of the third solid is 0% of the dry weight of the first acetic acid-producing sludge;

4) introducing the gas G produced in step 3) into a mixture of municipal sewage and the second acetic acid-producing sludge prepared in Example 1 in a reactor R2 for performing anaerobic culture and solid-liquid separation to obtain a second liquid L2 and a second solid S2, where, the concentration of the second acetic acid-producing sludge in the mixture is 500 mg/L; and conditions of anaerobic culture are that a pH value is 6 and time is 1 hour; and

5) mixing the first liquid L1 with the second liquid L2 in a reactor R3, measuring the concentration of ammonia nitrogen and a phosphate salt, adding a magnesium salt, adding ammonium chloride or sodium phosphate as needed to make a molar ratio of magnesium ions to ammonium ions to phosphate ions in the R3 reach 1:1:1 to adjusting the pH value to be 8, and performing stirring for 5 minutes and solid-liquid separation, where a precipitate is a fertilizer containing nitrogen and phosphorus, and a supernatant liquid is a liquid containing acetic acid. At the same time, the first solid S1, the second solid S2 and humic acid were thoroughly mixed and dried to obtain a third solid P. An added amount of humic acid was 10% of the total dry weight of the first solid S1 and the second solid S2, and the drying temperature was 20° C. In this example, the third solid obtained was not added in step 3).

Compared with Comparative Example 1, the yield of acetic acid can be increased by 157% without adding the third solid P in the present invention.

A first acetic acid-producing sludge and a second acetic acid-producing sludge obtained in Example 2 were used in Example 16;

a first acetic acid-producing sludge and a second acetic acid-producing sludge obtained in Example 3 were used in Example 17;

a first acetic acid-producing sludge and a second acetic acid-producing sludge obtained in Example 4 were used in Example 18;

a first acetic acid-producing sludge and a second acetic acid-producing sludge obtained in Example 5 were used in Example 19;

a first acetic acid-producing sludge and a second acetic acid-producing sludge obtained in Example 6 were used in Example 20;

a first acetic acid-producing sludge and a second acetic acid-producing sludge obtained in Example 7 were used in Example 21;

a first acetic acid-producing sludge and a second acetic acid-producing sludge obtained in Example 8 were used in Example 22;

a first acetic acid-producing sludge and a second acetic acid-producing sludge obtained in Example 9 were used in Example 23;

a first acetic acid-producing sludge and a second acetic acid-producing sludge obtained in Example 10 were used in Example 24;

a first acetic acid-producing sludge and a second acetic acid-producing sludge obtained in Example 11 were used in Example 25;

a first acetic acid-producing sludge and a second acetic acid-producing sludge obtained in Example 12 were used in Example 26;

a first acetic acid-producing sludge and a second acetic acid-producing sludge obtained in Example 13 were used in Example 27;

a first acetic acid-producing sludge and a second acetic acid-producing sludge obtained in Example 14 were used in Example 28; other steps were the same as those in Example 15. Specific parameters and results are shown in the following table.

TABLE 2
Parameters and results of Examples 15-28 and Comparative Examples 1-15

| | | | in step 3) | | | |
|---|---|---|---|---|---|---|
| | in step 1) | | | | Proportion of | |
| | Content of | | | | the added amount | |
| | solids in a | | Volume ratio of | | of the third | |
| | mixture of | | the first acetic | | solid in the dry | step 4) |
| | the urban | | acid-producing | | weight of the | Concentration |
| | wet garbage | in step 2) | sludge to the | Conditions | first acetic acid- | of sludge in a |
| | and water | Hydrolysis | urban wet | of anaerobic | producing sludge | mixture |
| | (mg/L) | conditions | garbage | culture | (%) | (mg/L) |
| Example 15 | 20 | pH value 8, | 8:100 | pH value 6, | 0 | 500 |
| | | temperature | | temperature | | |
| | | 5° C. and | | 20° C. and | | |
| | | time 1 hour | | time 1 day | | |
| Example 16 | 180 | pH value 8, | 8:100 | pH value 6, | 5 | 500 |
| | | temperature | | temperature | | |
| | | 5° C. and | | 20° C. and | | |
| | | time 1 hour | | time 1 day | | |
| Example 17 | 20 | pH value 8, | 8:100 | pH value 6, | 8 | 500 |
| | | temperature | | temperature | | |
| | | 5° C. and | | 20° C. and | | |
| | | time 1 hour | | time 1 day | | |
| Example 18 | 40 | pH value 8, | 8:100 | pH value 6, | 13 | 500 |
| | | temperature | | temperature | | |
| | | 5° C. and | | 20° C. and | | |
| | | time 1 hour | | time 1 day | | |
| Example 19 | 20 | pH value 8, | 8:100 | pH value 6, | 0 | 500 |
| | | temperature | | temperature | | |
| | | 5° C. and | | 20° C. and | | |
| | | time 1 hour | | time 1 day | | |
| Example 20 | 20 | pH value 8, | 8:100 | pH value 6, | 8 | 500 |
| | | temperature | | temperature | | |
| | | 5° C. and | | 20° C. and | | |
| | | time 1 hour | | time 1 day | | |
| Example 21 | 180 | pH value 8, | 8:100 | pH value 6, | 5 | 500 |
| | | temperature | | temperature | | |
| | | 5° C. and | | 20° C. and | | |
| | | time 1 hour | | time 1 day | | |
| Example 22 | 180 | pH value 10, | 8:100 | pH value 12, | 70 | 7000 |
| | | temperature | | temperature | | |
| | | 80° C. and | | 80° C. and | | |
| | | time 4 days | | time 12 days | | |
| Example 23 | 50 | pH value 8, | 8:100 | pH value 7, | 15 | 7000 |
| | | temperature | | temperature | | |
| | | 20° C. and | | 30° C. and | | |
| | | time 1 day | | time 3 days | | |
| Example 24 | 90 | pH value 10, | 8:100 | pH value 9, | 40 | 4000 |
| | | temperature | | temperature | | |
| | | 50° C. and | | 40° C. and | | |
| | | time 2 days | | time 6 days | | |
| Example 25 | 70 | pH value 9, | 8:100 | pH value 9, | 30 | 4000 |
| | | temperature | | temperature | | |
| | | 50° C. and | | 40° C. and | | |
| | | time 1.5 days | | time 5 days | | |
| Example 26 | 20 | pH value 8, | 8:100 | pH value 7, | 40 | 1000 |
| | | temperature | | temperature | | |
| | | 20° C. and | | 30° C. and | | |
| | | time 1 day | | time 3 days | | |
| Example 27 | 100 | pH value 8, | 8:100 | pH value 7, | 20 | 1300 |
| | | temperature | | temperature | | |
| | | 30° C. and | | 30° C. and | | |
| | | time 2 days | | time 3 days | | |
| Example 28 | 50 | pH value 9, | 8:100 | pH value 9, | 25 | 6000 |
| | | temperature | | temperature | | |
| | | 25° C. and | | 35° C. and | | |
| | | time 4 days | | time 2 days | | |

| | | | | | step 6) | | | |
|---|---|---|---|---|---|---|---|---|
| | | | | | Proportion of | | | |
| | | | | | the added amount | | | |
| | | | | | of humic acid in | | | |
| | | | | | the total dry | | Comparative | |
| | step 4) | | | | weight of the | | Example | |
| | Conditions | step 5) | | | first solid and | Drying | Content of | |
| | of anaerobic | Adjustment | Stirring | | the second solid | temperature | the urban | |
| | culture | of pH | time | | (%) | (° C.) | wet garbage | Results |
| Example 15 | pH value 6 | 8 | 5 | min | 10 | 20 | Comparative | 157 |
| | and time 1 | | | | | | Example 1: | |
| | hour | | | | | | 20 g/L | |
| Example 16 | pH value 6 | 8 | 5 | min | 10 | 20 | Comparative | 269 |
| | and time 1 | | | | | | Example 2: | |
| | hour | | | | | | 180 g/L | |
| Example 17 | pH value 6 | 8 | 5 | min | 10 | 20 | Comparative | 196 |
| | and time 1 | | | | | | Example 3: | |
| | hour | | | | | | 20 g/L | |
| Example 18 | pH value 6 | 8 | 5 | min | 10 | 20 | Comparative | 288 |
| | and time 1 | | | | | | Example 3: | |
| | hour | | | | | | 40 g/L | |
| Example 19 | pH value 6 | 8 | 5 | min | 10 | 20 | Comparative | 176 |
| | and time 1 | | | | | | Example 4: | |
| | hour | | | | | | 20 | |
| Example 20 | pH value 6 | 8 | 5 | min | 10 | 20 | Comparative | 257 |
| | and time 1 | | | | | | Example 5: | |
| | hour | | | | | | 20 | |
| Example 21 | pH value 6 | 8 | 5 | min | 10 | 20 | Comparative | 314 |
| | and time 1 | | | | | | example 6: | |
| | hour | | | | | | 180 | |
| Example 22 | pH value 8 | 10 | 50 | min | 100 | 120 | Comparative | 383 |
| | and time 1 | | | | | | example 7: | |
| | hour | | | | | | 180 | |
| Example 23 | pH value 7 | 8 | 10 | min | 20 | 30 | Comparative | 270 |
| | and time 1 | | | | | | Example 8: | |
| | hour | | | | | | 50 | |
| Example 24 | pH value 7 | 9 | 20 | min | 40 | 65 | Comparative | 614 |
| | and time 1 | | | | | | Example 9: | |
| | hour | | | | | | 90 | |
| Example 25 | pH value 7 | 10 | 50 | min | 30 | 30 | Comparative | 453 |
| | and time 1 | | | | | | Example 10: | |
| | hour | | | | | | 70 | |
| Example 26 | pH value 8 | 8 | 30 | min | 25 | 60 | Comparative | 298 |
| | and time 1 | | | | | | Example 11: | |
| | hour | | | | | | 20 | |
| Example 27 | pH value 7 | 9 | 15 | min | 30 | 100 | Comparative | 418 |
| | and time 1 | | | | | | example 12: | |
| | hour | | | | | | 100 | |
| Example 28 | pH value 7 | 9 | 20 | min | 50 | 120 | Comparative | 347 |
| | and time 1 | | | | | | Example 13: | |
| | hour | | | | | | 50 | |

It can be seen from Table 2 that compared with corresponding comparative examples using traditional methods, the yield of acetic acid in urban wet garbage can be increased by 157% or above by using the methods in examples of the present invention to treat the urban wet garbage. Compared with a treatment method without addition of the third solid, the yield of acetic acid can be increased by 1.4 times or above by adding the third solid into the hydrolysate in step 3) and the first acetic acid-producing sludge.

### Comparative Example 1

In this comparative example, a traditional treatment method for urban wet garbage includes the following steps:

1) mixing the urban wet garbage with a particle size of 0.1-1 mm with water to obtain a mixture, where a concentration of the urban wet garbage in the mixture is 20 g/L; and performing oil extraction to obtain an oil-extracted mixture; and 2) directly performing anaerobic culture on the oil-extracted mixture at a pH of 7 and a temperature of 25° C. for 6 days without adding a first acetic acid-producing sludge, a second acetic acid-producing sludge, a magnesium salt and humic acid.

Only the concentration of the urban wet garbage in the mixture in Comparative Examples 2-14 is different from that in Comparative Example 1, and others are the same as those in Example 1. The concentration of the urban wet garbage in the mixture in Comparative Examples 2-14 is shown in Table 2.

The above examples only exemplarily illustrate the principles and effects of the present invention, but are not used to limit the present invention. Any person skilled in the art may make modifications or changes on the foregoing examples without departing from the spirit and scope of the present invention. Therefore, all equivalent modifications or changes made by a person of ordinary skill in the art without departing from the spirit and technical idea of the present invention shall be covered by the claims of the present invention.

What is claimed is:

1. A high-value treatment system or method for urban wet garbage, wherein the urban wet garbage is used as a raw material, and the method comprises the following steps:
1) mixing the urban wet garbage with water and performing oil extraction to obtain an oil-extracted mixture;
2) mixing the oil-extracted mixture with an alkali for performing a hydrolysis reaction to obtain a hydrolysate;
3) performing anaerobic culture on the hydrolysate and a first acetic acid-producing sludge, collecting a produced gas and performing solid-liquid separation after culture is completed to obtain a first liquid and a first solid;
4) introducing the gas into a mixture of municipal sewage and a second acetic acid-producing sludge for performing anaerobic culture and solid-liquid separation to obtain a second liquid and a second solid; and
5) mixing the first liquid with the second liquid, adding a magnesium salt to adjust a pH value, and performing stirring and solid-liquid separation, wherein a precipitate is a fertilizer containing nitrogen and phosphorus, and a supernatant liquid is a liquid containing acetic acid; mixing the first solid, the second solid and humic acid, and performing drying to obtain a third solid,
wherein the first acetic acid-producing sludge is an acclimated sludge capable of converting glucose into acetic acid; and
the second acetic acid-producing sludge is an acclimated sludge capable of converting carbon dioxide and hydrogen into acetic acid.

2. The high-value treatment system or method for urban wet garbage according to claim 1, wherein an acclimation process of the first acetic acid-producing sludge comprises the following steps: adding glucose into a mixture of sludge and municipal sewage, and performing anaerobic culture at a pH of 6-11 and a temperature of 20° C. to 80° C. to obtain the first acetic acid-producing sludge;
and/or, an acclimation process of the second acetic acid-producing sludge comprises the following steps: introducing hydrogen and carbon dioxide into a mixture of sludge and municipal sewage, and performing anaerobic culture at a pH of 5-9 and a temperature of 20° C. to 50° C. to obtain the second acetic acid-producing sludge.

3. The high-value treatment system or method for urban wet garbage according to claim 2, wherein the acclimation process of the first acetic acid-producing sludge comprises three periods:
in the first period, a content of solids in the mixture is 3800-4500 mg/L;
and/or, in the first period, based on a total volume of the sludge, the municipal sewage and the glucose, a concentration of the glucose is 600 mgCOD/L to 1000 mg/L;
and/or, the first period is 3-7 days;
and/or, in the second period, the concentration of the glucose is maintained to be 1000-1400 mgCOD/L per day;
and/or, the second period is 8-12 days;
and/or, in the third period, the concentration of the glucose is daily increased by 80-100 mgCOD/L;
and/or, in the third period, acetic acid is also added, and a concentration of the acetic acid is maintained to be 30-70 mgCOD/L per day;
and/or, the third period is 30-35 days.

4. The high-value treatment system or method for urban wet garbage according to claim 1, wherein in step 1), a particle size of the urban wet garbage is 0.1-1 mm;
and/or, a content of solids in a mixture formed by mixing the urban wet garbage with water is 20-180 g/L;
and/or, an oil content of the oil-extracted mixture is lower than 3%.

5. The high-value treatment system or method for urban wet garbage according to claim 1, wherein in step 2), the alkali is sodium hydroxide, and conditions of the hydrolysis reaction are that a pH value is 8-12 and a temperature is 5° C. to 80° C.

6. The high-value treatment system or method for urban wet garbage according to claim 1, wherein in step 3), a volume ratio of the first acetic acid-producing sludge to the urban wet garbage is (6-10):100;
and/or, conditions of anaerobic culture are that a pH value is 6-12 and a temperature is 20° C. to 80° C.

7. The high-value treatment system or method for urban wet garbage according to claim 1, wherein in step 3), the third solid is also added into the hydrolysate and the first acetic acid-producing sludge, and an added amount of the third solid is not higher than 70% of a dry weight of the first acetic acid-producing sludge.

8. The high-value treatment system or method for urban wet garbage according to claim 1, wherein in step 4), based on a total volume of the mixture, a concentration of the second acetic acid-producing sludge is 500-7000 mg/L;
and/or, conditions of anaerobic culture are that a pH value is 6-8.

9. The high-value treatment system or method for urban wet garbage according to claim 1, wherein in step 5), the magnesium salt is magnesium chloride;
and/or, the pH value is adjusted to 8-10;
and/or, a stirring time is 5-50 minutes;
and/or, after the magnesium salt is added, an ammonia nitrogen salt and/or a phosphate salt is also added.

10. The high-value treatment system or method for urban wet garbage according to claim 1, wherein an added amount of humic acid is 10% to 100% of a total dry weight of the first solid and the second solid;
and/or, a drying temperature is 20° C. to 120° C.
